(19) United States
(12) Patent Application Publication
(10) Pub. No.: US 2024/0091269 A1
(43) Pub. Date: Mar. 21, 2024

(54) TREATMENT OF BIPOLAR DISORDER USING MESENCHYMAL STEM CELLS AND MODIFICATION OF MESENCHYMAL STEM CELLS

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); James VELTMEYER, Oceanside, CA (US); Kalina O'CONNOR, Oceanside, CA (US)

(73) Assignee: Therapeutic Solutions International, Inc., Oceanside, CA (US)

(21) Appl. No.: 18/470,304

(22) Filed: Sep. 19, 2023

Related U.S. Application Data
(60) Provisional application No. 63/408,017, filed on Sep. 19, 2022.

(51) Int. Cl. A61K 35/28; A61P 25/24

(52) U.S. Cl. CPC A61K 35/28; A61P 25/24

(57) ABSTRACT

The invention discloses the utilization of mesenchymal stem cells, exosomes from mesenchymal stem cells, conditioned media from mesenchymal stem cells, apoptotic bodies from mesenchymal stem cells, and modified mesenchymal stem cells for treatment of bipolar disorder. In one embodiment mesenchymal stem cells isolated from umbilical cord tissue are treated carbon monoxide at a concentration sufficient to induce activation of heme-oxygenase I and infused into a patient at risk or suffering from bipolar disorder.

### CROSS-REFERENCE TO RELATED APPLICATIONS

This application claims priority to U.S. Provisional Application No. 63/408,017, titled “Treatment of Bipolar Disorder Using Mesenchymal Stem Cells and Modification of Mesenchymal Stem Cells”, filed Sep. 19, 2022, which is hereby incorporated by reference in its entirety.

### FIELD OF THE INVENTION

The teachings herein relate to new treatments for bipolar disorder utilizing regenerative stem cells, such as mesenchymal stem cells and modified mesenchymal stem cells.

### BACKGROUND OF THE INVENTION

Mental illness takes a considerable toll on our society. Both the major depressive disorder (MDD) and the bipolar disorder (BD) are characterized by mood changes and are therefore referred to as affective disorders. Major depressive disorder is characterized by recurrent episodes of low mood and energy levels. Bipolar disorder is characterized by recurrent and alternating episodes of mood and energy-level disturbances, which are increased on some occasions, for example on mania or hypomania, and decreased on others, for example, on depression. In both major depressive disorder and bipolar disorder, changes in mood are often separated by periods of normal mood, known as euthymia. The major depressive disorder affects more women than men and its overall lifetime prevalence is 16%. In contrast, the bipolar disorder affects men and women equally, is associated with an earlier age of onset compared to the major depressive disorder, and its prevalence is 4-5-fold lower.

Although mania and hypomania are the most recognizable characteristics of the bipolar disorder, depression is its most frequent clinical presentation. Therefore, the patients suffering from a bipolar disorder are much more likely to present to clinicians when they are depressed, especially in outpatient settings. Unfortunately, the clinical presentation of a patient with bipolar disorder when depressed may not differ from that of a patient suffering from a major depressive disorder. This may explain why almost 40% of bipolar disorder patients are initially misdiagnosed with major depressive disorder and why the average delay for patients suffering from a bipolar disorder to be correctly diagnosed is of approximately 7.5 years.

Currently there are no consistent treatments to address underlying causes of bipolar disorder.

### SUMMARY

Preferred embodiments are directed to methods of preventing or treating bipolar disorder comprising administration of a regenerative cell population.

Preferred methods include embodiments wherein said bipolar disorder is associated with depression.

Preferred methods include embodiments wherein the depression is associated with lack of motivation.

Preferred methods include embodiments wherein said bipolar disorder is associated with augmented Th17 cells compared to age matched controls

Preferred methods include embodiments wherein said bipolar disorder is associated with augmented Th9 cells compared to age matched controls

Preferred methods include embodiments wherein said bipolar disorder is associated with augmented Th1 cells compared to age matched controls

Preferred methods include embodiments wherein said Th1 cells are found in peripheral blood

Preferred methods include embodiments wherein said Th1 cells are found in the bone microenvironment.

Preferred methods include embodiments wherein said Th1 cells are found in the brain.

Preferred methods include embodiments wherein said Th9 cells have higher activity as compared to an age-matched healthy control.

Preferred methods include embodiments wherein said Th9 cells are found in peripheral blood.

Preferred methods include embodiments wherein said Th9 cells are found in the bone microenvironment.

Preferred methods include embodiments wherein said Th9 cells are found in the brain.

Preferred methods include embodiments wherein said increase in Th9 cells is associated with enhanced interleukin-1 beta activity as compared to an age-matched healthy control.

Preferred methods include embodiments wherein said interleukin-1 beta is found in peripheral blood.

Preferred methods include embodiments wherein said interleukin-1 beta is found in the bone microenvironment.

Preferred methods include embodiments wherein said interleukin-1 beta is found in the brain.

Preferred methods include embodiments wherein said increased Th9 cells is associated with enhanced interleukin-6 activity as compared to an age-matched healthy control.

Preferred methods include embodiments wherein said interleukin-6 is found in peripheral blood.

Preferred methods include embodiments wherein said interleukin-6 is found in the bone microenvironment.

Preferred methods include embodiments wherein said interleukin-6 is found in the brain.

Preferred methods include embodiments wherein said increased Th9 cells is associated with enhanced interleukin-8 activity as compared to an age-matched healthy control.

Preferred methods include embodiments wherein said interleukin-8 is found in peripheral blood.

Preferred methods include embodiments wherein said interleukin-8 is found in the bone microenvironment.

Preferred methods include embodiments wherein said interleukin-8 is found in the osteoblasts.

Preferred methods include embodiments wherein said increased Th9 cells is associated with enhanced interleukin-11 activity as compared to an age-matched healthy control.

Preferred methods include embodiments wherein said interleukin-11 is found in peripheral blood.

Preferred methods include embodiments wherein said interleukin-11 is found in the bone microenvironment.

Preferred methods include embodiments wherein said interleukin-11 is found in the osteoblasts.

Preferred methods include embodiments wherein said increased Th9 activity is associated with enhanced interleukin-15 activity as compared to an age-matched healthy control.

Preferred methods include embodiments wherein said interleukin-15 is found in peripheral blood.

Preferred methods include embodiments wherein said interleukin-15 is found in the bone microenvironment.

Preferred methods include embodiments wherein said interleukin-15 is found in the osteoblasts.

Preferred methods include embodiments wherein said increased Th9 activity is associated with enhanced interleukin-17 activity as compared to an age-matched healthy control.

Preferred methods include embodiments wherein said interleukin-17 is found in peripheral blood.

Preferred methods include embodiments wherein said interleukin-17 is found in the bone microenvironment.

Preferred methods include embodiments wherein said interleukin-17 is found in the osteoblasts.

Preferred methods include embodiments wherein said increased Th9 activity is associated with enhanced interleukin-18 activity as compared to an age-matched healthy control.

Preferred methods include embodiments wherein said interleukin-18 is found in peripheral blood.

Preferred methods include embodiments wherein said interleukin-18 is found in the bone microenvironment.

Preferred methods include embodiments wherein said interleukin-18 is found in the osteoblasts.

Preferred methods include embodiments wherein said increased Th9 activity is associated with enhanced interleukin-33 activity as compared to an age-matched healthy control.

Preferred methods include embodiments wherein said interleukin-33 is found in peripheral blood.

Preferred methods include embodiments wherein said interleukin-33 is found in the bone microenvironment.

Preferred methods include embodiments wherein said interleukin-33 is found in the osteoblasts.

Preferred methods include embodiments wherein said regenerative cell is selected from either alone or in combination from a group comprising of: stem cells, committed progenitor cells, and differentiated cells and optionally are treated with carbon monoxide at a concentration sufficient to stimulate expression of HIF-1 alpha and/or heme oxygenase.

Preferred methods include embodiments wherein said stem cells are selected from a group comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

Preferred methods include embodiments wherein said embryonic stem cells are totipotent.

Preferred methods include embodiments wherein said embryonic stem cells express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Preferred methods include embodiments wherein said cord blood stem cells are multipotent and capable of differentiating into endothelial, smooth muscle, and neuronal cells.

Preferred methods include embodiments wherein said cord blood stem cells are identified based on expression of one or more antigens selected from a group comprising: SSEA-3, SSEA-4, CD9, CD34, c-kit, OCT-4, Nanog, and CXCR-4

Preferred methods include embodiments wherein said cord blood stem cells do not express one or more markers selected from a group comprising of: CD3, CD34, CD45, and CD11b.

Preferred methods include embodiments wherein said placental stem cells are isolated from the placental structure.

Preferred methods include embodiments wherein said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

Preferred methods include embodiments wherein said bone marrow stem cells comprise of bone marrow mononuclear cells.

Preferred methods include embodiments wherein said bone marrow stem cells are selected based on the ability to differentiate into one or more of the following cell types: endothelial cells, smooth muscle cells, and neuronal cells.

Preferred methods include embodiments wherein said bone marrow stem cells are selected based on expression of one or more of the following antigens: CD34, c-kit, flk-1, Stro-1, CD105, CD73, CD31, CD146, vascular endothelial-cadherin, CD133 and CXCR-4.

Preferred methods include embodiments wherein said bone marrow stem cells are enriched for expression of CD133.

Preferred methods include embodiments wherein said amniotic fluid stem cells are isolated by introduction of a fluid extraction means into the amniotic cavity under ultrasound guidance.

Preferred methods include embodiments wherein said amniotic fluid stem cells are selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1.

Preferred methods include embodiments wherein said amniotic fluid stem cells are selected based on lack of expression of one or more of the following antigens: CD34, CD45, and HLA Class II.

Preferred methods include embodiments wherein said neuronal stem cells are selected based on expression of one or more of the following antigens: RC-2, 3CB2, BLB, Sox-2hh, GLAST, Pax 6, nestin, Muashi-1, NCAM, A2B5 and prominin.

Preferred methods include embodiments wherein said circulating peripheral blood stem cells are characterized by ability to proliferate in vitro for a period of over 3 months.

Preferred methods include embodiments wherein said circulating peripheral blood stem cells are characterized by expression of CD34, CXCR4, CD117, CD113, and c-met.

Preferred methods include embodiments wherein said circulating peripheral blood stem cells lack substantial expression of differentiation associated markers.

Preferred methods include embodiments wherein said differentiation associated markers are selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

Preferred methods include embodiments wherein said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred methods include embodiments wherein said mesenchymal stem cells do not express substantial levels of HLA-DR, CD117, and CD45.

Preferred methods include embodiments wherein said mesenchymal stem cells are derived from a group selected of: bone marrow, adipose tissue, umbilical cord blood, placental tissue, peripheral blood mononuclear cells, differentiated embryonic stem cells, and differentiated progenitor cells.

Preferred methods include embodiments wherein said germinal stem cells express markers selected from a group comprising of: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1.

Preferred methods include embodiments wherein said adipose tissue derived stem cells express markers selected from a group comprising of CD13, CD29, CD44, CD63, CD73, CD90, CD166, Aldehyde dehydrogenase (ALDH), and ABCG2.

Preferred methods include embodiments wherein said adipose tissue derived stem cells are a population of purified mononuclear cells extracted from adipose tissue capable of proliferating in culture for more than 1 month.

Preferred methods include embodiments wherein said exfoliated teeth derived stem cells express markers selected from a group comprising of: STRO-1, CD146 (MUC18), alkaline phosphatase, MEPE, and bFGF.

Preferred methods include embodiments wherein said hair follicle stem cells express markers selected from a group comprising of: cytokeratin 15, Nanog, and Oct-4.

Preferred methods include embodiments wherein said hair follicle stem cells are capable of proliferating in culture for a period of at least one month.

Preferred methods include embodiments wherein said hair follicle stem cells secrete one or more of the following proteins when grown in culture: basic fibroblast growth factor (bFGF), endothelin-1 (ET-1) and stem cell factor (SCF).

Preferred methods include embodiments wherein said dermal stem cells express markers selected from a group comprising of: CD44, CD13, CD29, CD90, and CD105.

Preferred methods include embodiments wherein said dermal stem cells are capable of proliferating in culture for a period of at least one month.

Preferred methods include embodiments wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Preferred methods include embodiments wherein said reprogrammed stem cells are selected from a group comprising of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions.

Preferred methods include embodiments wherein said nuclear transfer comprises introducing nuclear material to a cell substantially enucleated, said nuclear material deriving from a host whose genetic profile is sought to be dedifferentiated.

Preferred methods include embodiments wherein said cytoplasmic transfer comprises introducing cytoplasm of a cell with a dedifferentiated phenotype into a cell with a differentiated phenotype, such that said cell with a differentiated phenotype substantially reverts to a dedifferentiated phenotype.

Preferred methods include embodiments wherein said DNA demethylating agent is selected from a group comprising of: 5-azacytidine, psammaplin A, and zebularine.

Preferred methods include embodiments wherein said histone deacetylase inhibitor is selected from a group comprising of: valproic acid, trichostatin-A, trapoxin A and depsipeptide.

The side population cells of claim 46, wherein said cells are identified based on expression multidrug resistance transport protein (ABCG2) or ability to efflux intracellular dyes such as rhodamine-123 and or Hoechst 33342.

The side population cells of claim 85, wherein said cells are derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

Preferred methods include embodiments wherein said committed progenitor cells are selected from a group comprising of: endothelial progenitor cells, neuronal progenitor cells, and hematopoietic progenitor cells.

Preferred methods include embodiments wherein said committed endothelial progenitor cells are purified from the bone marrow.

Preferred methods include embodiments wherein said committed endothelial progenitor cells are purified from peripheral blood.

Preferred methods include embodiments wherein said committed endothelial progenitor cells are purified from peripheral blood of a patient whose committed endothelial progenitor cells are mobilized by administration of a mobilizing agent or therapy.

Preferred methods include embodiments wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA)reductase inhibitors and small molecule antagonists of SDF-1.

Preferred methods include embodiments wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow.

Preferred methods include embodiments wherein said committed endothelial progenitor cells express markers selected from a group comprising of: CD31, CD34, AC133, CD146 and flk1.

Preferred methods include embodiments wherein said committed hematopoietic cells are purified from the bone marrow.

Preferred methods include embodiments wherein said committed hematopoietic progenitor cells are purified from peripheral blood.

Preferred methods include embodiments wherein said committed hematopoietic progenitor cells are purified from peripheral blood of a patient whose committed hematopoietic progenitor cells are mobilized by administration of a mobilizing agent or therapy.

Preferred methods include embodiments wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA)reductase inhibitors and small molecule antagonists of SDF-1.

Preferred methods include embodiments wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow.

Preferred methods include embodiments wherein said committed hematopoietic progenitor cells express the marker CD133.

Preferred methods include embodiments wherein said committed hematopoietic progenitor cells express the marker CD34.

Preferred methods include embodiments wherein an antioxidant is administered at a therapeutically sufficient concentration to a patient in need thereof.

Preferred methods include embodiments wherein said antioxidant is selected from a group comprising of: ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol.

Preferred methods include embodiments wherein an NF-kappa B inhibitor is administered prior to, subsequently with, or after administration of regenerative cells.

Preferred methods include embodiments wherein said NF-kappa B inhibitor is administered directly into the ovary.

Preferred methods include embodiments wherein said NF-kappa B inhibitor is administered systemically.

Preferred methods include embodiments wherein said NF-kappa B inhibitor is administered using bone targeting technology.

Preferred methods include embodiments wherein said bone targeting technology are liposomes.

Preferred methods include embodiments wherein said bone targeting technology are immunoliposomes.

Preferred methods include embodiments wherein said bone targeting technology are nanoparticles.

Preferred methods include embodiments wherein said bone targeting technology are quantum dots.

### BRIEF DESCRIPTION OF THE DRAWINGS

FIG. 1 is a bar graph showing the effects of bone marrow MSC, adipose MSC, and CampbellCell on reducing bipolar behavior in mice in an open-field test.

FIG. 2 is a bar graph showing the effects of bone marrow MSC, adipose MSC, and CampbellCell on reducing TNF-alpha levels in mice.

FIG. 3 is a bar graph showing the effects of bone marrow MSC, adipose MSC, and CampbellCell on reducing IL-17 levels in mice.

### DETAILED DESCRIPTION OF THE INVENTION

The invention provides the use of various regenerative cells for treatment of bipolar disorder and/or prevention.

It is to be understood that this invention is not limited to the particular methodology, protocols, animal species or genera, and reagents described, as such may vary. It is also to be understood that the terminology used herein is for the purpose of describing particular embodiments only, and is not intended to limit the scope of the present invention that will be limited only by the appended claims.

As used herein the term “subject”, refers to an animal, preferably a mammal, and most preferably a human both male and female, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of one or more of the signs or symptoms of the disease or disorder being treated.

The term “prophylactically effective amount” is intended to mean that amount of a pharmaceutical drug that will prevent or reduce the risk of occurrence of the biological or medical event that is sought to be prevented of a tissue, a system, animal or human that is being sought by a researcher, veterinarian, medical doctor or other clinician.

The term “pharmaceutically acceptable salts or amides” shall mean non-toxic salts or amides of the compounds employed in this invention together with conditioned media from regenerative cells which are generally prepared by reacting the free acid with a suitable organic or inorganic base. Examples of such salts include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride: edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, Iactobionate, laurate, malate, maleate, mandelate, mesylate, methylbromide, methylnitrate, methylsulfate, mucate, napsylate, nitrate, oleate, oxalate, pamaote, palmitate, panthothenate, phosphate/diphosphate, polygalacturonate, potassium, salicylate, sodium, stearate, subacetate, succinate, militate, tartrate, teoclate, tosylate, triethiodide, valerate.

Therefore, the term “a patient in need of treatment” as used herein will refer to any subject or patient who currently has or may develop any of the above syndromes or disorders, including any mood disorder which can be treated by antidepressant medication, or any other disorder in which the patient's present clinical condition or prognosis could benefit from the administration of one or more compounds of Formula (1) alone or in combination with another therapeutic intervention including but not limited to another medication.

The term “treating” or “treatment” as used herein, refers to any indicia of success in, the prevention or amelioration of an injury, pathology or condition of bipolar disorder and modification or symptoms of bipolar disorder, including any objective or subjective parameter such as abatement; remission; diminishing of symptoms or making the injury, pathology, or condition more tolerable to the patient; slowing in the rate of degeneration or decline or worsening of the illness; making the final point of worsening less debilitating; or improving a subject's physical or mental well-being. The treatment or amelioration of symptoms can be based on objective or subjective parameters; including the results of a physical examination, neurological examination, and/or psychiatric evaluations. Accordingly, the term “treating” or “treatment” includes the administration of the compounds or agents of the present invention, for treatment of any form of bipolar disorder in both males and females. In some instances, treatment with the compounds of the present mention will done in combination with other compounds to prevent, inhibit, or arrest the progression of bipolar disorder.

The term “therapeutic effect” as used herein, refers to the effective improvement in or reduction of symptoms of bipolar disorder. The term “a therapeutically effective amount” as used herein means, a sufficient amount of one or more of the compounds of the invention to produce a therapeutic effect, as defined above, in a subject or patient in need of such bipolar disorder treatment.

The terms “subject” or “patient” are used herein interchangeably and as used herein mean any mammal including but not limited to human beings including a human patient or subject to which the compositions of the invention can be administered. The term mammals include human patients, both male and female and non-human primates, as well as experimental animals such as rabbits, rats, mice, and other animals.

In embodiments of the invention where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342. Without being bound to theory, cells possessing this property often express the multidrug resistance transport protein ABCG2, and are known for enhanced regenerative ability compared to cells which do not possess this efflux mechanism. In other embodiments cord blood cells are purified for certain therapeutic properties based on expression of markers. In one particular embodiment, cord blood cells are purified for the phenotype of endothelial precursor cells. Said precursors, or progenitor cells express markers such as CD133, and/or CD34. Said progenitors may be purified by positive or negative selection using techniques such as magnetic activated cell sorting (MACS), affinity columns, FACS, panning, or by other means known in the art. Cord blood derived endothelial progenitor cells may be administered directly into the target tissue for ED, or may be administered systemically. Another variation of this embodiment is the use of differentiation of said endothelial precursor cells in vitro, followed by infusion into a patient. Verification for endothelial differentiation may be performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

Certain desired activities can be endowed onto said cord blood stem cells prior to administration into the patient. In one specific embodiment cord blood cells may be “activated” ex vivo by a brief culture in hypoxic conditions in order to upregulate nuclear translocation of the HIF-1 transcription factor and endow said cord blood cells with enhanced angiogenic potential. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours, more preferably from 13 hours to 59 hours and more preferably around 48 hours. Assessment of angiogenic, and other desired activities useful for the practice of the current invention, can be performed prior to administration of said cord blood cells into the patient. Assessment methods are known in the art and include measurement of angiogenic factors, ability to support viability and activity of cells associated with erectile function, as well as ability to induce regeneration of said cellular components associated with erectile function.

In addition to induction of hypoxia, other therapeutic properties can be endowed unto cord blood stem cells through treatment ex vivo with factors such as de-differentiating compounds, proliferation inducing compounds, or compounds known to endow and/or enhance cord blood cells to possess properties useful for the practice of the current invention. In one embodiment cord blood cells are cultured with an inhibitor of the enzyme GSK-3 in order to enhance expansion of cells with pluripotent characteristics while not increasing the rate of differentiation. In another embodiment, cord blood cells are cultured in the presence of a DNA methyltransferase inhibitor such as 5-azacytidine in order to endow a “de-differentiation” effect. In another embodiment cord blood cells are cultured in the presence of a differentiation agent that skews said cord blood stem cells to generate enhance numbers of cells which are useful for treatment of bipolar disorder after said cord blood cells are administered into a patient. For example, cord blood cells may be cultured in estrogen for a brief period so that subsequent to administration, an increased number of follicular cells generated in the patient in need thereof.

In contrast to cord blood stem cells, placental stem cells may be purified directly from placental tissues, said tissues including the chorion, amnion, and villous stroma. In another embodiment of the invention, placental tissue is mechanically degraded in a sterile manner and treated with enzymes to allow dissociation of the cells from the extracellular matrix. Such enzymes include, but not restricted to trypsin, chymotrypsin, collagenases, elastase and/or hylauronidase. Suspension of placental cells are subsequently washed, assessed for viability, and may either be used directly for the practice of the invention by administration either locally or systemically. Alternatively, cells may be purified for certain populations with increased biological activity. Purification may be performed using means known in the art, and described above for purification of cord blood stem cells, or may be achieved by positive selection for the following markers: SSEA3, SSEA4, TRA1-60, TRA1-81, c-kit, and Thy-1. In some situations it will be desirable to expand cells before introduction into the human body. Expansion can be performed by culture ex vivo with specific growth factors [80, 81]. The various embodiments of the invention described above for cord blood and embryonic stem cells can also be applied for placental stem cells.

In some embodiments of the invention, administration of “platelet rich plasma” is performed in order to augment regenerative activities of stem cells. Administration may be locally with the stem cells or may be in a place different from the stem cells. “Platelet rich plasma” (PRP) as described herein is a blood plasma that has been enriched with platelets. As a concentrated source of autologous platelets, PRP contains and releases several different growth factors and other cytokines that stimulate healing of bone and soft tissue. Components of PRP may include but are not limited to platelet-derived growth factor, transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, and combinations thereof. PRP may be prepared by collection of the patient's whole blood (that is anticoagulated with citrate dextrose) before undergoing two stages of centrifugation designed to separate the PRP aliquot from platelet-poor plasma and red blood cells. In humans, a typical baseline blood platelet count may range from about 150,000 to about 450,000 platelets per.mu.l of blood, or about 200,000 platelets per.mu.l of blood. Therapeutic PRP may concentrate platelets in plasma by about five-fold. As such, PRP platelet count in PRP may range from about 750,000 to about 2.25.times.10.sup.6 platelets per.mu.l of PRP, or about 1.times.10.sup.6 platelets per.mu.l of blood. The PRP may then be used to prepare human platelet lysate. Compositions of the present disclosure may comprise platelet plasma compositions from PRP, HPL, or combinations thereof, and either platelet plasma composition may be used to regenerate bone tissue for augmentation of fertility. Further, the platelet plasma composition may be used with or without concentrated bone marrow (BMAC). By way of example, when administered into bone tissue, about 0.05 to about 2.0 cc of platelet plasma composition may be used. Platelets are non-nucleated blood cells that as noted above are found in bone marrow and peripheral blood. In various embodiments of the present invention, the platelet plasma composition may be obtained by sequestering platelets from whole blood or bone marrow through centrifugation, for example into three strata: (1) platelet rich plasma; (2) platelet poor plasma; and (3) fibrinogen. When using platelets from one of the strata, e.g., the platelet rich plasma (PRP) from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires bone tissue regeneration. In some embodiments the lysate will act more rapidly than the PRP (or platelet poor plasma from bone marrow). Human platelet lysate may be formed from but not limited to PRP, pooled platelets from humans, and cultured megakaryocytes from stem cell expansion technology. In some embodiments, HPL is from a commercial source. In some embodiments, HPL is prepared in the laboratory from platelet rich plasma (PRP), pooled platelets from humans, or cultured megakaryocytes from stem cell expansion technology. Notably, platelet poor plasma that is derived from bone marrow has a greater platelet concentration than platelet rich plasma from blood, also known as platelet poor/rich plasma (“PP/RP” or “PPP”). PP/RP or PPP may be used to refer to platelet poor plasma derived from bone marrow, and in some embodiments, preferably PP/RP is used or PRP is used as part of the composition for disc regeneration. (By convention, the abbreviation PRP refers only to compositions derived from peripheral blood and PPP (or PP/RP) refers to compositions derived from bone marrow.) In various embodiments, the platelet plasma composition, which may or may not be in the form of a lysate, may serve one or more of the following functions: (1) to release/provide growth factors and cytokines for tissue regeneration; (2) to reduce inflammation; (3) to attract/mobilize cell signaling; (4) to initiate repair of damaged/atrophied bone tissue through fibroblast growth factors (FGF); (5) to stabilize extracellular matrix in the ovary; (6) to stimulate maturation of immature oocytes; (7) to stimulate revascularization of fibrotic tissue; and (8) to stimulate oocyte receptivity to spermatozoa. Additionally, by combining platelet therapy with stem cells, there can be synergy with respect to augmentation of fertility. In some embodiments in which the lysate is used, the cytokines may be concentrated in order to optimize their functional capacity. Concentration may be accomplished in two steps. First, blood may be obtained and concentrated to a volume that is 5-15% of what it was before concentration. Devices that may be used include but are not limited to a hemofilter or a hemo-concentrator. For example, 60 cc of blood may be concentrated down to 6 cc. Next, the concentrated blood may be filtered to remove water. This filtering step may reduce the volume further to 33%-67% (e.g., approximately 50%) of what it was prior to filtration. Thus, by way of example for a concentration product of 6 cc, one may filter out water so that one obtains a product of approximately 3 cc. When the platelet rich plasma, platelet poor plasma and fibrinogen are obtained from blood, they may for example be obtained by drawing 20-500 cc of peripheral blood, 40-250 cc of peripheral blood, or 60-100 cc of peripheral blood. The amount of blood that one should draw will depend on the extent of bone tissue degeneration. In some embodiments, a method of generation of said PRP may be used according to U.S. Pat. No. 9,011,929, which is incorporated by reference herein in its entirety. In essence, a method may comprise separating PRP from whole blood by collecting whole blood from an animal or patient into a vacuum test tube containing sodium citrate, and primarily centrifuging the collected whole blood; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid; and collecting the PRP concentrated in a bottom layer by another blunt needle; mixing the PRP collected from the separating step with a calcium chloride solution by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at room temperature before mixing; and mixing the mixture of the PRP and the calcium chloride solution with the type I collagen, in an opaque phase, four times by another three-way connector. In an exemplary embodiment of the disclosure, a method may comprise separating the PRP from whole blood, wherein the separating step further comprises the steps of: collecting 10 ml of the whole blood from an animal or patient into a vacuum test tube containing 3.2% sodium citrate, and primarily centrifuging the collected whole blood at 1,750-1,900 g for 3 to 5 minutes; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid at 4,500-5,000 g for 4 to 6 minutes; and collecting the PRP concentrated in a bottom layer by another blunt needle; mixing 1 mL of the PRP collected from the separating step with a calcium chloride solution with a concentration of 0.30-0.55 mg/mL by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at a room temperature for 15 to 30 minutes before mixing; and mixing the mixture of the PRP and the calcium chloride solution with the type I collagen with a concentration of 20-50 mg/mL, in an opaque phase, four times by another three-way connector. The term “platelet-rich plasma” or “PRP” as used herein is a broad term which is used in its ordinary sense and is a concentration of platelets greater than the peripheral blood concentration suspended in a solution of plasma, or other excipient suitable for administration to a human or non-human animal including, but not limited to, isotonic sodium chloride solution, physiological saline, normal saline, dextrose 5% in water, dextrose 10% in water, Ringer solution, lactated Ringer solution, Ringer lactate, Ringer lactate solution, and the like. PRP compositions may be an autologous preparation from whole blood taken from the subject to be treated or, alternatively, PRP compositions may be prepared from a whole blood sample taken from a single donor source or from whole blood samples taken from multiple donor sources. In general, PRP compositions comprise platelets at a platelet concentration that is higher than the baseline concentration of the platelets in whole blood. In some embodiments, PRP compositions may further comprise WBCs at a WBC concentration that is higher than the baseline concentration of the WBCs in whole blood. As used herein, baseline concentration means the concentration of the specified cell type found in the patient's blood which would be the same as the concentration of that cell type found in a blood sample from that patient without manipulation of the sample by laboratory techniques such as cell sorting, centrifugation or filtration. Where blood samples are obtained from more than one source, baseline concentration means the concentration found in the mixed blood sample from which the PRP is derived without manipulation of the mixed sample by laboratory techniques such as cell sorting, centrifugation or filtration. In some embodiments, PRP compositions comprise elevated concentrations of platelets and WBCs and lower levels of RBCs and hemoglobin relative to their baseline concentrations. In some embodiments of PRP composition, only the concentration of platelets is elevated relative to the baseline concentration. Some embodiments of PRP composition comprise elevated levels of platelets and WBCs compared to baseline concentrations. In some embodiments, PRP compositions comprise elevated concentrations of platelets and lower levels of neutrophils relative to their baseline concentrations. Some embodiments of PRP composition comprise elevated levels of platelets and neutrophil-depleted WBCs compared to their baseline concentrations. In some embodiments of PRP, the ratio of lymphocytes and monocytes to neutrophils is significantly higher than the ratios of their baseline concentrations. The PRP formulation may include platelets at a level of between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration may be between about 4 and about 6 times the baseline. Typically, a microliter of whole blood comprises at least 140,000 to 150,000 platelets and up to 400,000 to 500,000 platelets. The PRP compositions may comprise about 500,000 to about 7,000,000 platelets per microliter. In some instances, the PRP compositions may comprise about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, about 2,500,000 to about 5,000,000, or about 5,000,000 to about 7,000,000 platelets per microliter. The WBC concentration is typically elevated in PRP compositions. For example, the WBC concentration may be between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, or higher. The WBC count in a microliter of whole blood is typically at least 4,100 to 4,500 and up to 10,900 to 11,000. The WBC count in a microliter of the PRP composition may be between about 8,000 and about 10,000; about 10,000 and about 15,000; about 15,000 and about 20,000; about 20,000 and about 30,000; about 30,000 and about 50,000; about 50,000 and about 75,000; and about 75,000 and about 100,000. Among the WBCs in the PRP composition, the concentrations may vary by the cell type but, generally, each may be elevated. In some variations, the PRP composition may comprise specific concentrations of various types of white blood cells. The relative concentrations of one cell type to another cell type in a PRP composition may be the same or different than the relative concentration of the cell types in whole blood. For example, the concentrations of lymphocytes and/or monocytes may be between about 1.1 and about 2 times baseline, about 2 and about 4 times baseline, about 4 and about 6 times baseline, about 6 and about 8 times baseline, or higher. In some variations, the concentrations of the lymphocytes and/or the monocytes may be less than the baseline concentration. The concentrations of eosinophils in the PRP composition may be less than baseline, about 1.5 times baseline, about 2 times baseline, about 3 times baseline, about 5 times baseline, or higher. In whole blood, the lymphocyte count is typically between 1,300 and 4,000 cells per microliter, but in other examples, the lymphocyte concentration may be between about 5,000 and about 20,000 per microliter. In some instances, the lymphocyte concentration may be less than 5,000 per microliter or greater than 20,000 per microliter. The monocyte count in a microliter of whole blood is typically between 200 and 800. In the PRP composition, the monocyte concentration may be less than about 1,000 per microliter, between about 1,000 and about 5,000 per microliter, or greater than about 5,000 per microliter. The eosinophil concentration may be between about 200 and about 1,000 per microliter elevated from about 40 to 400 in whole blood. In some variations, the eosinophil concentration may be less than about 200 per microliter or greater than about 1,000 per microliter. In certain variations, the PRP composition may contain a specific concentration of neutrophils. The neutrophil concentration may vary between less than the baseline concentration of neutrophils to eight times than the baseline concentration of neutrophils. In some embodiments, the PRP composition may include neutrophils at a concentration of 50-70%, 30-50%, 10-30%, 5-10%, 1-5%, 0.5-1%, 0.1-0.5% of levels of neutrophils found in whole blood or even less. In some embodiments, neutrophil levels are depleted to 1% or less than that found in whole blood. In some variations, the neutrophil concentration may be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. One microliter of whole blood typically comprises 2,000 to 7,500 neutrophils. In some variations, the PRP composition may comprise neutrophils at a concentration of less than about 1,000 per microliter, about 1,000 to about 5,000 per microliter, about 5,000 to about 20,000 per microliter, about 20,000 to about 40,000 per microliter, or about 40,000 to about 60,000 per microliter. In some embodiments, neutrophils are eliminated or substantially eliminated. Means to deplete blood products, such as PRP, of neutrophils is known and discussed in U.S. Pat. No. 7,462,268, which is incorporated herein by reference. Several embodiments are directed to PRP compositions in which levels of platelets and white blood cells are elevated compared to whole blood and in which the ratio of monocytes and/or lymphocytes to neutrophils is higher than in whole blood. The ratio of monocytes and/or lymphocytes to neutrophils may serve as an index to determine if a PRP formulation may be efficaciously used as a treatment for a particular disease or condition. PRP compositions where the ratio of monocytes and/or lymphocytes to neutrophils is increased may be generated by either lowering neutrophils levels, or by maintaining neutrophil levels while increasing levels of monocytes and/or lymphocytes. Several embodiments relate to a PRP formulation that contains 1.01 times, or higher, baseline platelets in combination with a 1.01 times, or higher, baseline white blood cells with the neutrophil component depleted at least 1% from baseline. In some embodiments, the PRP compositions may comprise a lower concentration of red blood cells (RBCs) and/or hemoglobin than the concentration in whole blood. The RBC concentration may be between about 0.01 and about 0.1 times baseline, about 0.1 and about 0.25 times baseline, about 0.25 and about 0.5 times baseline, or about 0.5 and about 0.9 times baseline. The hemoglobin concentration may be depressed and in some variations may be about 1 g/dl or less, between about 1 g/dl and about 5 g/dl, about 5 g/dl and about 10 g/dl, about 10 g/dl and about 15 g/dl, or about 15 g/dl and about 20 g/dl. Typically, whole blood drawn from a male patient may have an RBC count of at least 4,300,000 to 4,500,000 and up to 5,900,000 to 6,200,000 per microliter while whole blood from a female patient may have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women. In some embodiments, PRP compositions contain increased concentrations of growth factors and other cytokines. In several embodiments, PRP compositions may include increased concentrations of one or more of: platelet-derived growth factor, transforming growth factor beta, fibroblast growth factor, insulin-like growth factor, insulin-like growth factor 2, vascular endothelial growth factor, epidermal growth factor, interleukin-8, keratinocyte growth factor, and connective tissue growth factor. In some embodiments, the platelets collected in PRP are activated by thrombin and calcium chloride to induce the release of these growth factors from alpha granules. In some embodiments, a PRP composition is activated exogenously with thrombin and/or calcium to produce a gel that can be applied to an area to be treated. The process of exogenous activation, however, results in immediate release of growth factors. Other embodiments relate to activation of PRP via in vivo contact with collagen containing tissue at the treatment site. The in vivo activation of PRP results in slower growth factor release at the desired site. In certain embodiments of the invention, the PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or location of the tissue damage and/or the MI, the availability of prepared PRP, or any suitable combination of factors. Any suitable amount of blood may be collected. For example, about 1 cc to about 150 cc of blood or more may be drawn. More specifically, about 27 cc to about 110 cc or about 27 cc to about 55 cc of blood may be withdrawn. In some embodiments, the blood may be collected from a patient who may be presently suffering, or who has previously suffered from, connective tissue damage and/or an MI. PRP made from a patient's own blood may significantly reduce the risk of adverse reactions or infection. In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area. The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume. In some examples, the blood may then be centrifuged using a gravitational platelet system, such as the Cell Factor Technologies GPS System® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained. As the PRP composition comprises activated platelets, active agents within the platelets are released. These agents include, but are not limited to, cytokines (e.g., IL-1B, IL-6, TNF-A), chemokines (e.g., ENA-78 (CXCL8), IL-8 (CXCL8), MCP-3 (CCL7), MIP-1A (CCL3), NAP-2 (CXCL7), PF4 (CXCL4), RANTES (CCL5)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-.beta. 1, 2, and 3, and VEGF). Said PRP may be used to treat autologous regenerative cells prior to administration of said cells for stimulation of ovary regeneration and/or prevention of immunologically mediated abortions. One type of autologous regenerative cells are adipose stromal vascular fraction cells. Said stromal vascular fraction cells are obtained by the following steps; a) Using aseptic technique and with local anesthesia, the infraumbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37.degree. C. shaking water bath for one hour then centrifuged for 5 min at 300 rcf; g) Following centrifugation, the stromal cells are resuspended within Isolyte in separate sterile 50 mL centrifuge tubes; h) The tubes are centrifuged for 5 min. at 300 rcf and the Isolyte is removed, leaving cell pellet; i) The pellets are resuspended in 40 ml of Isolyte, centrifuged again for 5 min at 300rcf. The supernatant is again be removed; j) The cell pellets are combined and filtered through 100.quadrature.m cell strainers into a sterile 50 ml centrifuge tube and centrifuged for 5 min at 300rcf and the supernatant removed, leaving the pelleted adipose stromal cells. Means of combining PRP and SVF are known in the literature and incorporated by reference. In some embodiments, the neutrophils are depleted by at least 5%, in some embodiments, the neutrophils are depleted by at least 10%, in some embodiments, the neutrophils are depleted by at least 15%, in some embodiments, the neutrophils are depleted by at least 20%, in some embodiments, the neutrophils are depleted by at least 25%, in some embodiments, the neutrophils are depleted by at least 30%, in some embodiments, the neutrophils are depleted by at least 35%, in some embodiments, the neutrophils are depleted by at least 40%, in some embodiments, the neutrophils are depleted by at least 45%, in some embodiments, the neutrophils are depleted by at least 50%, in some embodiments, the neutrophils are depleted by at least 55%, in some embodiments, the neutrophils are depleted by at least 60%, in some embodiments, the neutrophils are depleted by at least 65%, in some embodiments, the neutrophils are depleted by at least 70%, in some embodiments, the neutrophils are depleted by at least 75%, in some embodiments, the neutrophils are depleted by at least 80%, in some embodiments, the neutrophils are depleted by at least 85%, in some embodiments, the neutrophils are depleted by at least 90%, in some embodiments, the neutrophils are depleted by at least 95%, in some embodiments, the neutrophils are depleted by at least 95%. In some embodiments, the neutrophils in the platelet rich plasma are substantially removed.] Administration of PRP intraovarially may be performed using methods known in the art. Exemplary publications, which are incorporated by reference for guidance in the practice of the invention are provided. In some embodiments of the invention, autologous regenerative cells such as adipose stromal vascular fraction cells, and/or bone marrow mononuclear cells are administered together with platelet rich plasma and/or platelet lysate.

Bone marrow stem cells may be used either freshly isolated, purified, or subsequent to ex vivo culture. A typical bone marrow harvest for collecting starting material for practicing one embodiment of the invention involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest”. Said aspirate is used as a starting material for purification of cells with ability to prevent bipolar disorder In one specific embodiment bone marrow mononuclear cells are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass), or the Fresenius AS 104 and the Fresenius AS TEC 104 (Fresenius, Bad Homburg, Germany) separators. In addition to injection of mononuclear cells, purified bone marrow subpopulations may be used. Additionally, ex vivo expansion and/or selection may also be utilized for augmentation of desired biological properties for use in treatment of bipolar disorder. The various embodiments of the invention described above for cord blood and embryonic stem cells can also be applied for bone marrow stem cells.

Amniotic fluid is routinely collected during amniocentesis procedures. One method of practicing the current invention is utilizing amniotic fluid derived stem cells for treatment of bipolar disorder. In one embodiment amniotic fluid mononuclear cells are utilized therapeutically in an unpurified manner. Said amniotic fluid stem cells are administered either locally or systemically in a patient suffering from bipolar disorder. In other embodiments amniotic fluid stem cells are substantially purified based on expression of markers such as SSEA-3, SSEA4, Tra-1-60, Tra-1-81 and Tra-2-54, and subsequently administered. In other embodiments cells are cultured, as described in US patent application #20050054093, expanded, and subsequently infused into the patient. Amniotic stem cells are described in the following references. One particular aspect of amniotic stem cells that makes them amenable for use in practicing certain aspects of the current invention is their bi-phenotypic profile as being both mesenchymal and neural progenitors.

A wide variety of stem cells are known to circulate in the periphery. These include multipotent, pluripotent, and committed stem cells. In some embodiments of the invention mobilization of stem cells is induced in order to increase the number of circulating stem cells, so that harvesting efficiency is increased. Said mobilization allows for harvest of cells with desired properties for practice of the invention without the need to perform bone marrow puncture. A variety of methods to induce mobilization are known. Methods such as administration of cytotoxic chemotherapy, for example, cyclophosphamide or 5-fluoruracil are effective but not preferred in the context of the current invention due to relatively unacceptable adverse events profile. Suitable agents useful for mobilization include: granulocyte colony stimulating factor (G-CSF), granulocyte-macrophage colony stimulating factor (GM-CSF), interleukin 1 (IL-1), interleukin 3 (IL-3), stem cell factor (SCF, also known as steel factor or kit ligand), vascular endothelial growth factor (VEGF), Flt-3 ligand, platelet-derived growth factor (PDGF), epidermal growth factor (EGF), fibroblast growth factor-1 (FGF-1), fibroblast growth factor-2 (FGF-2), thrombopoietin (TPO), interleukin-11 (IL-11), insulin-like growth factor-1 (IGF-1), megakaryocyte growth and development factor (MGDF), nerve growth factor (NGF), hyperbaric oxygen, and 3-hydroxy-3-methyl glutaryl coenzyme A (HMG CoA)reductase inhibitors. The various embodiments of the invention described above for cord blood and embryonic stem cells can also be applied for circulating peripheral blood stem cells.

In a preferred embodiment, donors (either autologous or allegeneic) are mobilized by administration of G-CSF (filgrastim: neupogen) at a concentration of 10 ug/kg/day by subcutaneous injection for 2-7 days, more preferably 4-5 days. Peripheral blood mononuclear cells are collected using an apheresis device such as the AS104 cell separator (Fresenius Medical). 1-40×109 mononuclear cells are collected, concentrated and injected into the area of penile flow occlusion in an intramuscular manner. Alternatively, cells may be injected systemically, or in an area proximal to the region of penile blood flow occlusion. Identification of such occlusion is routinely known in the art and includes the use of penile ultrasonometry. Variations of this procedure may include steps such as subsequent culture of cells to enrich for various populations known to possess angiogenic and/or neurogenic, and/or anti-atrophy Additionally cells may be purified for specific subtypes before and/or after culture. Treatments can be made to the cells during culture or at specific timepoints during ex vivo culture but before infusion in order to generate and/or expand specific subtypes and/or functional properties.

In one embodiment mesenchymal cells are generated through culture. For example, U.S. Pat. No. 5,486,359 describes methods for culturing such and expanding mesenchymal stem cells, as well as providing antibodies for use in detection and isolation. U.S. Pat. No. 5,942,225 teaches culture techniques and additives for differentiation of such stem cells which can be used in the context of the present invention to produce increased numbers of cells with angiogenic and/or follicogenic capability. Although U.S. Pat. No. 6,387,369 teaches use of mesenchymal stem cells for regeneration of cardiac tissue, we believe that in accordance with published literature stem cells generated through these means are actually angiogenically potent and therefore may be utilized in the context of the current invention for treatment/amelioration of bipolar disorder. Without being bound to a specific theory or mechanism of action, it appears that mesenchymal stem cells induce angiogenesis through production of factors such as vascular endothelial growth factor, hepatocyte growth factor, adrenomedullin, and insulin-like growth factor-1.

Mesenchymal stem cells are classically obtained from bone marrow sources for clinical use, although this source may have disadvantages because of the invasiveness of the donation procedure and the reported decline in number of bone marrow derived mesenchymal stem cells during aging. Alternative sources of mesenchymal stem cells include adipose tissue, placenta, scalp tissue and cord blood. A recent study compared mesenchymal stem cells from bone marrow, cord blood and adipose tissue in terms of colony formation activity, expansion potential and immunophenotype. It was demonstrated that all three sources produced mesenchymal stem cells with similar morphology and phenotype. Ability to induce colony formation was highest using stem cells from adipose tissue and interestingly in contrast to bone marrow and adipose derived mesenchymal cells, only the cord blood derived cells lacked ability to undergo adipocyte differentiation. Proliferative potential was the highest with cord blood mesenchymal stem cells which were capable of expansion to approximately 20 times, whereas cord blood cells expanded an average of 8 times and bone marrow derived cells expanded 5 times [94]. Accordingly, one skilled in the art will understand that mesenchymal stem cells for use with the present invention may be selected upon individual patient characteristics and the end result sought. For example, if autologous mesenchymal stem cells are available in the form of adipocyte-derived cells, it will be useful to utilize this source instead of allogeneic cord-blood derived cells. Alternatively, cord blood derived mesenchymal stem cells may be more advantageous for use in situations where autologous cells are not available, and expansion is sought.

Adipose derived stem cells express markers such as CD9; CD29 (integrin beta 1); CD44 (hyaluronate receptor); CD49d,e (integrin alpha 4, 5); CD55 (decay accelerating factor); CD105 (endoglin); CD106 (VCAM-1); CD166 (ALCAM). These markers are useful not only for identification but may be used as a means of positive selection, before and/or after culture in order to increase purity of the desired cell population. In terms of purification and isolation, devices are known to those skilled in the art for rapid extraction and purification of cells adipose tissues. U.S. Pat. No. 6,316,247 describes a device which purifies mononuclear adipose derived stem cells in an enclosed environment without the need for setting up a GMP/GTP cell processing laboratory so that patients may be treated in a wide variety of settings. One embodiment of the invention involves attaining 10-200 ml of raw lipoaspirate, washing said lipoaspirate in phosphate buffered saline, digesting said lipoaspirate with 0.075% collagenase type I for 30-60 min at 37° C. with gentle agitation, neutralizing said collagenase with DMEM or other medium containing autologous serum, preferably at a concentration of 10% v/v, centrifuging the treated lipoaspirate at approximately 700-2000 g for 5-15 minutes, followed by resuspension of said cells in an appropriate medium such as DMEM. Cells are subsequently filtered using a cell strainer, for example a 100 μm nylon cell strainer in order to remove debris. Filtered cells are subsequently centrifuged again at approximately 700-2000 g for 5-15 minutes and resuspended at a concentration of approximately 1×106/cm2 into culture flasks or similar vessels. After 10-20 hours of culture non-adherent cells are removed by washing with PBS and remaining cells are cultured at similar conditions as described above for culture of cord blood derived mesenchymal stem cells. Upon reaching a concentration desired for clinical use, cells are harvested, assessed for purity and administered in a patient in need thereof as described above. The various embodiments of the invention described above for cord blood and embryonic stem cells can also be applied for adipose derived stem cells. Tooth derived stem cells have been recently identified as a source of pluripotent stem cells with ability to differentiate into endothelial, neural, and bone structures. Said pluripotent stem cells have been termed “stem cells from human exfoliated deciduous teeth” (SHED). One of the embodiments of the current invention involves utilization of this novel source of stem cells for the treatment of bipolar disorder. In one embodiment of the invention, SHED cells are administered systemically or locally into a patient with bipolar disorder at a concentration and frequency sufficient for induction of therapeutic effect. SHED cells can be purified and used directly, certain subpopulations may be concentrated, or cells may be expanded ex vivo under distinct culture conditions in order to generate phenotypes desired for maximum therapeutic effect. Growth and expansion of SHED has been previously described by others. In one particular method, exfoliated human deciduous teeth are collected from 7- to 8-year-old children, with the pulp extracted and digested with a digestive enzyme such as collagenase type I. Concentrations necessary for digestion are known and may be, for example 1-5 mg/ml, or preferable around 3 mg/ml. Additionally dispase may also be used alone or in combination, concentrations of dispase may be 1-10 mg/ml, preferably around 4 mg/ml. Said digestion is allowed to occur for approximately 1 h at 37° C. Cells are subsequently washed and may be used directly, purified, or expanded in tissue culture. The various embodiments of the invention described above for cord blood and embryonic stem cells can also be applied for exfoliated teeth stem cells.

### WORKING EXAMPLES

### Example 1: CampbellCell Reduces Bipolar Behavior Induced by Ouabian in the Open Field Test

Ouabain 0.1 mg/kg was administered to BALB/c mice to induce bipolar disorder. Animals where treated with control cells (splenocytes), bone marrow MSC, adipose MSC and CampbellCell at 500,000 cells per mouse intravenously 24 hours subsequent to Ouabain injection.

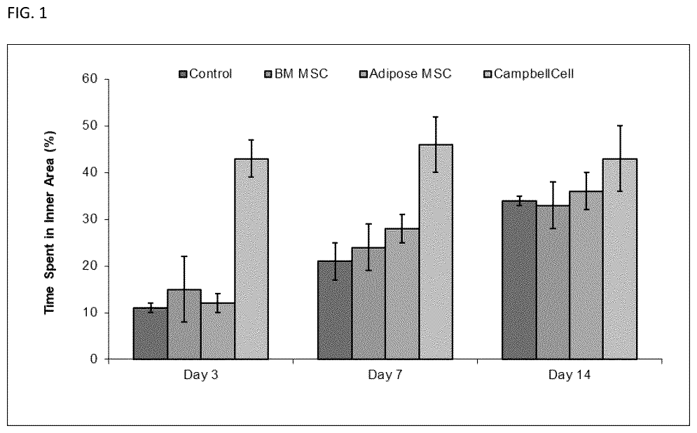

Bipolar behavior was analyzed at day 3, 7 and 14 after ouabain injection using an open-field test. An open-field test was performed for 5 min in a 50×50 cm area. Locomotion data were recorded and analyzed using a video tracking system. Less time spend in the inner area represents bipolar behavior. Results are shown in FIG. 1

### Example 2: CampbellCell Reduces Serum TNF-Alpha after Ouabain Injection

Ouabain 0.1 mg/kg was administered to BALB/c mice to induce bipolar disorder. Animals where treated with control cells (splenocytes), bone marrow MSC, adipose MSC and CampbellCell at 500,000 cells per mouse intravenously 24 hours subsequent to Ouabain injection.

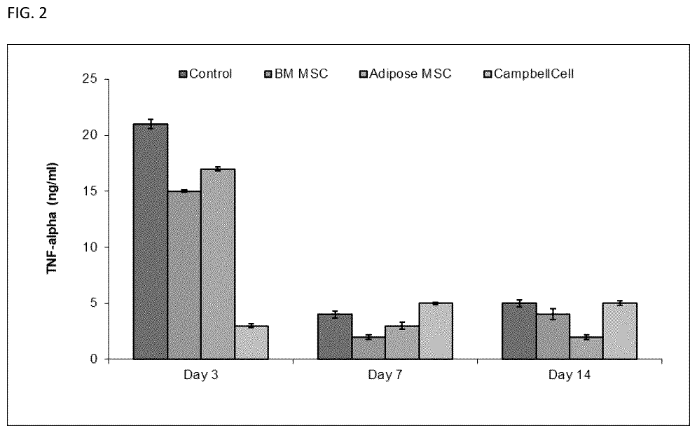

Plasma TNF-alpha was analyzed at day 3, 7 and 14 after ouabain injection by ELISA. Results are shown in FIG. 2.

### Example 3: CampbellCell Reduces Serum IL-17 after Ouabain Injection

Ouabain 0.1 mg/kg was administered to BALB/c mice to induce bipolar disorder. Animals where treated with control cells (splenocytes), bone marrow MSC, adipose MSC and CampbellCell at 500,000 cells per mouse intravenously 24 hours subsequent to Ouabain injection.

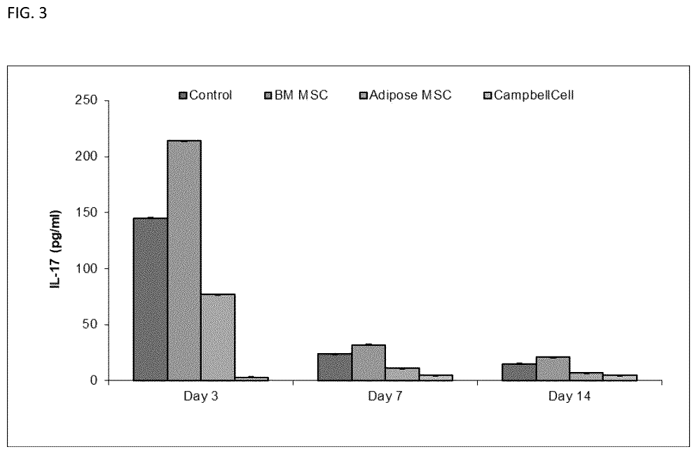

Plasma IL-17 was analyzed at day 3, 7 and 14 after ouabain injection by ELISA. Results are shown in FIG. 3.

### Example 4: Adoptive Transfer of Protection from Bipolar Pathology by T Cell Transfusion

Mice protected from bipolar pathology were sacrificed on day 21 and B cells, T cells, monocytes, and NK cells were sorted and transfused into naïve mice. Only transfusion with T cells resulted in reduction of bipolar pathology after adoptive transfer.

1. A method of preventing or treating bipolar disorder in a mammal comprising administration of a regenerative cell population to said mammal in an amount sufficient to ameliorate or prevent the symptoms of bipolar disorder.

2. The method of claim 1, wherein said bipolar disorder is associated with depression.

3. The method of claim 2, wherein the depression is associated with lack of motivation.

4. The method of claim 1, wherein said bipolar disorder is associated with augmented Th17 cells compared to age matched controls.

5. The method of claim 1, wherein said bipolar disorder is associated with augmented Th9 cells compared to age matched controls.

6. The method of claim 1, wherein said bipolar disorder is associated with augmented Th1 cells compared to age matched controls.

7. The method of claim 6, wherein said Th1 cells are found in peripheral blood.

8. The method of claim 6, wherein said Th1 cells are found in the bone microenvironment.

9. The method of claim 6, wherein said Th1 cells are found in the brain.

10. The method of claim 5, wherein said Th9 cells have higher activity as compared to an age-matched healthy control.

11. The method of claim 10, wherein said Th9 cells are found in peripheral blood.

12. The method of claim 10, wherein said Th9 cells are found in the bone microenvironment.

13. The method of claim 10, wherein said Th9 cells are found in the brain.

14. The method of claim 5, wherein said increase in Th9 cells is associated with enhanced interleukin-1 beta activity as compared to an age-matched healthy control.

15. The method of claim 14, wherein said interleukin-1 beta is found in peripheral blood.

16. The method of claim 14, wherein said interleukin-1 beta is found in the bone microenvironment.

17. The method of claim 14, wherein said interleukin-1 beta is found in the brain.

18. The method of claim 5, wherein said increased Th9 cells is associated with enhanced interleukin-6 activity as compared to an age-matched healthy control.

19. The method of claim 18, wherein said interleukin-6 is found in peripheral blood.

20. The method of claim 18, wherein said interleukin-6 is found in the bone microenvironment.
